(19) United States
(12) Patent Application Publication
(10) Pub. No.: US 2025/0352070 A1
(43) Pub. Date: Nov. 20, 2025

(54) SYSTEM, SENSOR DEVICE, AND METHOD FOR DETERMINING DIFFERENTIAL TEMPERATURE IN BODY PART

(71) Applicant: DAVION HEALTHCARE PLC, Limassol (CY)

(72) Inventor: Jack KAYE, Limassol (CY)

(73) Assignee: DAVION HEALTHCARE PLC, Limassol (CY)

(21) Appl. No.: 18/873,943

(22) PCT Filed: Jun. 14, 2023
(86) PCT No.: PCT/IB2023/056131

§ 371 Date: Dec. 11, 2024

(30) Foreign Application Priority Data
Jun. 14, 2022 (GB) 2208671.4

(51) Int. Cl. A61B 5/01; A61B 5/00; G16H 30/40

(52) U.S. Cl. CPC A61B 5/015; A61B 5/0077; A61B 5/4312; A61B 5/4387; A61B 5/44; A61B 5/6832; G16H 30/40; A61B 2562/0276

(57) ABSTRACT

A system for determining differential temperature in body part includes a sensor device(s) with a base having first side and second side opposite to first side; and temperature-sensitive element(s) being provided on first side of base, wherein when sensor device(s) in use, first side is in contact with body part of user for predefined time period and temperature-sensitive element(s) senses temperatures of different regions of body part; camera(s) configured to capture image(s) of first side, wherein image(s) captured within given time period from time of removal of contact between first side and body part; processor(s) communicably coupled to camera(s), wherein processor(s) configured to: identify, in image(s), image segment(s) representing temperature-sensitive element(s); recognize pixel values of pixels image segment(s); determine temperatures of different regions of body part, based on pixel values of pixels in image segment(s), temperatures of different regions of body part form differential temperature profile of body part.

### TECHNICAL FIELD

The present invention relates to a sensor device. In particular, though not exclusively, this invention relates to a system for determining the differential temperature in a body part, a sensor device, and a method for determining the differential temperature in a body part.

### BACKGROUND

Several techniques have been used to monitor body temperature of a person. Accurate monitoring of body temperature is crucial in order to determine the health of a person. A condition of high body temperature might indicate the presence of an infection. In some cases, a differential body temperature (having different temperatures at regions of one body part) at a specific body part may indicate the presence of a life-threatening disease. For example, the differential high temperature of a breast of a person indicates abnormalities in the breast of the person. As the abnormalities in the breast often produce additional heat through abnormal metabolic activity resulting in increased temperature of the breast. These abnormalities can lead to the development of potentially malignant tumour cells which can prove to be a fatal situation for the person. Several techniques are currently employed for the detection of malignant tumours in the beast in order to determine if the person's breast has been afflicted with cancer. Various techniques, include thermography, mammography, xerography, or the like. However, at the present, thermography is most widely used in the detection of breast cancer. Thermography is based on infra-red scanning of the surface of the breast and developing a thermogram which contains temperature information corresponding to the scanned surface. Since the temperature of malignant mammary tissues are usually higher than the temperature of normal tissues. However, this technique involves the use of expensive equipment and expert technicians or radiologists to analyse and interpret the thermogram.

In addition to the thermogram, other simpler devices have also been used to check temperature of the breast at home with ease. However, these devices also pose some challenges to user as their results are needed to be visually examined by the user which might severely affect accuracy and authenticity of the result. Moreover, these devices are often faced with a problem of usability as they are operational at a specific temperature range. Thus, these devices may fail to provide accurate results when used outside the specific temperature range. Furthermore, other limitations offered by conventional devices are the absence of any check while using or reusing the device, which severely compromises the accuracy and consistency of results obtained using the conventional device.

Therefore, in light of the foregoing discussion, there exists a need to overcome the aforementioned drawbacks associated with conventional systems and devices for monitoring the differential temperature of the breast of the user.

### SUMMARY OF THE INVENTION

A first aspect of the invention provides a system for determining the differential temperature in a body part, the system comprising: at least one sensor device comprising
a base having a first side and a second side opposite to the first side; and
at least one temperature-sensitive element provided on the first side of the base, wherein when the at least one sensor device is in use, the first side is in contact with the body part of a user for a predefined time period and the at least one temperature-sensitive element senses temperatures of different regions of the body part;
at least one camera configured to capture at least one image of the first side, wherein the at least one image is captured within a given time period from a time of removal of contact between the first side and the body part; and
at least one processor communicably coupled to the at least one camera, wherein the at least one processor is configured to:
   identify, in the at least one image, at least one image segment representing the at least one temperature-sensitive element;
   recognize pixel values of pixels in the at least one image segment; and
   determine the temperatures of different regions of the body part, based on the pixel values of pixels in the at least one image segment, wherein the temperatures of different regions of the body part form a differential temperature profile of the body part.

The at least one sensor device may be a wearable device, a non-wearable device, a device provided on a surface. is used to detect any potential abnormalities in the body part which typically possess a higher localised temperature higher than the parts of the body without abnormalities. The at least one sensor device functions by measuring the temperature difference of various regions of the body part to detect any potential abnormalities in that body part. Some abnormalities can lead to, or already be, tumours or cancerous tumours. Optionally, the at least one sensor device may be used in a posture comprising any one of: standing, sitting, lying down. It will be appreciated that teachings of the present disclosure are not limited to any particular body part. However, the at least one sensor device of the present disclosure can be worn on any part of the body to obtain an indication of a potential abnormality based upon the difference in temperature between two different portions of such body part.

Optionally, the body part may be one of: at least one breast, at least one portion of the skin, at least one testicle, at least one foot. A technical effect of determining differential temperatures of the aforementioned body parts is that these body parts show a difference in temperature when the user suffers from disease, and the at least one sensor device can be used in a non-invasive manner for early-stage detection of any disease. When the body part is the at least one breast, there could be an abnormality in the at least one breast when the user suffers from a breast-related disease, wherein said abnormality frequently produces additional heat. Such abnormality could be present in at least one particular portion of the at least one breast or the whole at least one breast. Examples of such breast-related diseases which produces abnormalities in the at least one breast, may include, but are not limited to, inflammatory breast cancer, mastitis, abscess, and fibrocystic changes. When the body part is at least one portion of the skin, there could be another abnormality in the at least one portion of the skin when the user suffers from skin-related disease, wherein said another abnormality frequently produces additional heat. Additionally, the first side of the at least one sensor device is made to be in contact with the at least one portion of the skin to determine a level of fatigue in underlying muscle of the at least one portion of the skin, and/or a potential rupture of muscle. Examples of such skin-related diseases which produces abnormalities in the at least one portion of the skin, may include, but are not limited to, inflammatory skin conditions, infections, vasculitis, and Raynaud's disease. When the body part is at least one testicle, there could be yet another abnormality in the at least one testicle when the user suffers from testicle-related disease, wherein said yet another abnormality frequently produces additional heat. Such yet another abnormality could be present in at least one another particular portion of the at least one testicle or the whole at least one testicle. Examples of such testicle-related diseases which produces abnormalities in the at least one testicle, may include, but are not limited to, varicocele, orchitis, hydrocele, testicular cancer, and epididymitis. When the body part is at least one foot, there could be still another abnormality in the at least one foot arising due to poor blood circulation, poor nerve function, inflammatory responses, when the user suffers from diseases, for example, such as Peripheral Arterial Disease (PAD), diabetes, peripheral neuropathy, and inflammatory arthritis. It will be appreciated that the usage of the system is not limited to the aforementioned body parts.

The at least one sensor device can be in contact with the body part of the user for a predefined time period. In an instance, when the body part is the at least one breast, the predefined time period may lie in a range of 5 minutes to 30 minutes. In another instance, when the body part is one of: the at least one portion of the skin, the at least one foot, the predefined time period may lie in a range of 5 minutes to 15 minutes. As an example, when the body part is one of: the at least one portion of the skin, the at least one foot, the predefined time period may be 10 minutes. In yet another instance, when the body part is the at least one testicle, the predefined time period may lie in a range of 5 minutes to 20 minutes. As an example, when the body part is the at least one testicle, the predefined time period may be 15 minutes.

Throughout the present disclosure, the term “base” refers to structural component on which the at least one sensor device is built. The term “first side” refers to a surface of the base, and the term “second side” refers to another surface of the base which is opposite to the first side. For example, the first side of the base may be the surface in contact with the body part and the second side may be another surface which is not in contact with the body part. The second side may be made of a soft material comprising at least one of: a logo, a branding, a design, a protective covering, and similar.

Throughout the present disclosure, the term “temperature-sensitive element” refers to a component that detects and measures temperature of the body part. Such measurement occurs upon contact of the sensor device with a body part, which for example may have regions of different temperatures. To measure the temperature of a body part or to measure different temperature of different regions of a body part, temperature-sensitive element is deigned such that it changes colour proportionally to the temperature of each region of a body part and therefore it creates a colour map of the said body part. This temperature measurement can be measured numerically, upon a prior calibration which assigns a colour to a temperature value, for example in Celsius. Such calibration may be added as a strip adhered to the sensor device (e.g. glued) or directly printed on to the device (for example at an edge). However, as explained below, preferably the numerical measurement is made by at least one software after a user captures the image of the temperature map. Optionally, the at least one temperature-sensitive element may be designed in such a manner that at least one of: an electrical property, a physical property, a chemical property changes in response to a change in the temperature of the body part. Optionally, the at least one temperature-sensitive element may be designed based on the body part with which the at least one sensor device is made to be in contact with. The first side, having the at least one temperature-sensitive element, stays in contact with the body part of the user, when in use. The at least one temperature-sensitive element may include one large temperature-sensitive element or a plurality of small temperature-sensitive elements, depending upon the application of the at least one sensor device. The at least one temperature-sensitive element senses the temperatures of the different regions of the body part.

Optionally, when the body part may be the at least one breast, the base of the at least one sensor device may have a hole, and the at least one temperature-sensitive element may at least partially surround the hole. The hole is provided in the at least one sensor device to accommodate a nipple region of each breast of the user, wherein the nipple region is placed through said hole. A technical effect of designing the at least one sensor device in such a manner such that a region of potential occurrence of an abnormality is covered by the at least one temperature-sensitive element at least partially or completely.

Optionally, the contact between the first side and the body part may be made using at least one of: an adhesive, a supporting element upon which the at least one sensor device is arranged and which is to be placed in contact with the body part, a tying means, a clamping means. A technical effect of making a contact between the first side and the body part is that any additional heat generated due to at least one of: the abnormality, the another abnormality, the yet another abnormality, the still another abnormality, can be measured to determine whether the body part is suffering from any disease or not. In an instance, when the contact is made using the adhesive, the adhesive may be any one of: provided as a layer on the first side of the at least one sensor device, applied, provided as a layer on the body part of the user to adhere the at least one sensor device to the body part. It will be appreciated that the adhesive is selected in such a manner that said adhesive is not harmful to the body part. Examples of the adhesive layer may include, but are not limited to, a medical grade adhesive, a liquid bandage adhesive, and an acrylic adhesive. For example, when the at least one body part is at least one portion of an underside of an arm, the at least one sensor device makes contact using the adhesive which may be provided on the first side of the base.

In another instance, the contact may be made using the supporting element. When in use, the at least one sensor device is arranged on the supporting means in such a manner that any one of: the body part is placed on the first side, the first side is placed on the body part. The supporting means may be used to facilitate the contact between the first side and the body part. This contact may be needed to be established using the supporting means when the body part may be hard to reach. As an example, when the body part is at least one breast, the supporting means may be a well-fitting bra. As another example, when the body part of at least one foot, the supporting means may be a pad on which the second side of the at least one sensor device is arranged so that the first side is in contact with at least one of: toes, heel of the at least one foot, wherein the pad can be used when the user is any one of: standing, sitting, sleeping.

In yet another instance, the contact may be made using the tying means. Herein, the at least one sensor device is tied securely to the body part in such a manner that the first side is in contact with the body part. Examples of such tying means may include, but are not limited to, a string, a belt, an elastic rope, a strap, and a Velcro strap. In still another instance, the contact may be made using the clamping means. Herein, the at least one sensor device is clamped securely to the body part in such a manner that the first side is in contact with the body part. Examples of such clamping means may include, but are not limited to, a screw clamp, a spring clamp, and a suction clamp.

The at least one camera could be a Red-Green-Blue (RGB) camera, a grayscale camera, or the like. The at least one camera captures the at least one image of the first side of the base. The at least one image is captured within the given time period upon removal of contact between the at least one sensor device from the body part. The given time period may lie in a range of 0-15 minutes. The given time period is determined based upon the at least one temperature-sensitive material used in the at least one sensor device. The given time period is determined such that a visual effect produced by the at least one temperature-sensitive material does not get diminished and the at least one image of the first side is captured properly by the at least one camera to further interpret results of the at least one sensor device. In an example, the at least one image is captured within 2 minutes after removal of the at least one sensor device from the body part.

Throughout the present disclosure, the term “processor” relates to a computational element that is operable to respond to and process instructions. The at least one processor, in operation, implements the method for determining the differential temperature in the body part. Furthermore, the term “processor” may refer to one or more individual processors, processing devices and various elements associated with a processing device that may be shared by other processing devices. Such processors, processing devices and elements may be arranged in various architectures for responding to and executing the steps of the method.

The at least one processor is communicatively coupled to the at least one camera via a communication network. It will be appreciated that the communication network may be wired, wireless, or a combination thereof. The communication network could be an individual network or a combination of multiple networks. Examples of the communication network may include, but are not limited to one or more of, Internet, a local network (such as, a TCP/IP-based network, an Ethernet-based local area network, an Ethernet-based personal area network, a Wi-Fi network, and the like), Wide Area Networks (WANs), Metropolitan Area Networks (MANs), a telecommunication network, and a short-range radio network (such as Bluetooth®).

The at least one processor is configured to execute at least one software application for implementing at least one processing task that the at least one processor is configured for. The at least one software application could be a single software application or a plurality of software applications. The at least one software application is preferably implemented as application for a mobile device such as a smartphone. The at least one software application reads and interprets results from the at least one sensor device. Such interpretation may made via comparison of the value (i.e., the colour) of a particular pixel or groups of pixels of the image with the expected pixel value (i.e., colour) for that represents the temperature of a body part.

Preferably, such interpretation is made by mapping each pixel of the image to identify its value, and subsequently, based on the calibration of the temperature sensitive material, retrieve the corresponding temperature of a particular pixel value through a corresponding characteristic of the colour, and assign temperature to the corresponding pixel value, therefore creating a map of temperature distribution of the skin of a body part that was previously in contact with the temperature sensitive material. Subsequently the at least one software identifies boundaries of regions of lower and higher temperatures by reading the corresponding pixel values within a given range. For example, values with a temperature difference lower than a fraction of a degree Celsius (for example, 0.25, 0.5, 0.75), or values within 1 degree Celsius may be considered as part of the same region. Such consideration solely depends on a previous calibration of the at least one software used. Once different regions are identified, the at least one software, may indicate that a region of a higher temperature is a indication of a potential anomaly. For example, this may be a region of a relatively higher concentration of pixels in a colour map (i.e., temperature map) with a relative temperature difference from neighbouring regions of 1 degree Celsius or 1.5 degree Celsius. Preferably, this may be a region of with a relative temperature difference from neighbouring regions of 2 degrees Celsius or higher.

The at least one software application could be an individual software application for an individual body part, or could be common software application for all the body parts. The at least one processor receives the at least one image of the first side of the at least one sensor device. The at least one image could be received by the at least one processor as a video, an image, a live stream, and the like. Preferably, the at least one image is received by the at least one processor via the communication network. The at least one processor identifies, the at least one image segment representing the at least one temperature-sensitive element of the at least one sensor device, in the at least one image. In other words, the at least one image is analysed for variations in colour (i.e., a colour gradient) of the at least one temperature-sensitive element, both prior to usage and when in use. The at least one processor identifies the at least one image segment by employing at least one image processing algorithm. Optionally, a given image processing algorithm is an object identification algorithm, a feature detection algorithm, and the like.

Post identification of the at least one image segment, the at least one processor is configured to recognize pixel values of pixels in the at least one image segment. The term “pixel value” of a pixel refers to colour information represented by the pixel. Herein, the colour information may be indicative of the colour gradient, which may be predefined and calibrated for differences in temperature of the body part. It is important to clarify that differences in temperature of a body may be a reference to differences in temperature of portions of a body part. For example, different portions (or different regions) of a person's breast may have different temperatures from each other. In such a case, if a person's breast is referred to as a body part, then different parts (portions or regions) of such breast would have temperatures that are different from each other.

Such calibration depends on an implementation of at least one of: the at least one temperature-sensitive material, a thickness of the base. The colour information may be expressed as Red-Green-Blue (RGB) colour component values, grayscale values, or similar. The Red-Green-Blue (RGB) colour component values of a given colour component in the at least one image segment may lie in a range of 0-256. The grayscale values of the at least one image segment may lie in a range of 0-1. As an exemplary implementation, when the system is in use and the body part is at least one foot (i.e., a left foot and a right foot), the at least one camera may capture an image of the at least one temperature-sensitive element. The at least one processor is configured to analyse different temperature for the body part, dissected into comparative sections (i.e., the at least one image segment) of each foot to provide an indication of the differential temperature the left foot and the right foot at comparative locations.

Furthermore, the at least one processor determines the temperatures of different regions of the body part. The at least one image has pixels of varying pixel values. Each of the pixel values of the at least one image determines the corresponding temperature of the body part. In an example, the at least one image segment may have three regions of varying colours, wherein for example, a first region may have an orange colour (243, 203, 23), a second region may have a pink colour (243, 122, 124) and a third region may have a yellow colour (243, 214, 10). Amongst these three regions, the region having the orange colour may be the region with a highest temperature, the region having the yellow may be the region with a lowest temperature amongst the three regions. In another example, the at least one image segment may have three regions. A first region may have a pixel value of 0.2, a second region may have a pixel value of 0.24 and a third region may have a pixel value of 0.8. Amongst three regions, the third region may be the region having the highest temperature indicating a potential abnormality in the corresponding region of the body part.

It will be appreciated that the at least one camera, communicably coupled to the at least one processor, that is able to execute a software application could be embodied through a singular device, for example, a user's camera smartphone and the like.

In this regard, when the at least one sensor device is worn by the user, the at least one temperature-sensitive element senses the temperatures of different regions of the body part with the help of the at least one temperature-sensitive material provided on the at least one sensor device. The at least one temperature-sensitive material may undergo colour transformation depending upon varying temperatures across regions of the body part. As an example, the at least one temperature-sensitive material may turn its colour from a blue colour to a pink colour as the temperature rises across the body part. As another example, the temperature-sensitive material may turn its state from solid to liquid. As yet another example, when a temperature-sensitive material of the at least one temperature-sensitive element is a thermochromic ink, said temperature-sensitive material may have a substantially dark colour, for example black, when the at least one sensor device is not in use, provided that the ambient temperature is lower than or close to (for example within 1 degree) of the lower limit of the range of operation of the temperature-sensitive material.

When the ambient temperature of the temperature sensitive is above the lower limit of the range of operation of the material, the material will adopt a different colour, which is proportional to the ambient temperature. Provided that ambient temperature measured at the ambient in which the temperature-sensitive material is to be used is lower than the higher limit of the range of operation, the material will still be able to show difference in temperatures between different regions of such material, and thus, it would be able to indicate the temperature differential of different regions of a body part when such temperature-sensitive material is in contact with such body part.

Therefore, when the at least one sensor device is in use, the temperature-sensitive material may have different colours at different temperatures within a predefined temperature range Therefore, the at least one image of the at least one temperature-sensitive element has varying colours across the at least one image segment. Therefore, the at least one processer is able to determine temperatures of different regions of the body part by analyzing the pixel values of pixels of the at least one image segment. The temperatures of different regions of the body part form the differential temperature profile of the body part. To increase the accuracy of the at least one sensor device, the user is instructed to avoid using the at least one sensor in a heated condition of the body, such as after exercise, after sunbath, or the like.

The system as disclosed above provides a kit to check for abnormalities in the body part of the user by determining differential temperatures in the body part. The system of the present disclosure is an early detection system that is safe, reliable, economical, accurate, and easier to use than conventional systems. The system is effectively used to routinely monitor for abnormalities in the body part and is intended to be used as an adjunct to other procedures, including established procedures, for the detection of diseases. For example, when the body part is the at least one breast, the established procedures are clinical breast examination and mammography. The system can be efficiently used for users of all ages.

The system for determining the differential temperature in a body part is technically superior to other conventional systems. The present system uses the software application to measure the differential temperature of the body part. The system can position temperature from the body part effectively to the software application. The software application significantly increases the accuracy and consistency of measurement obtained using the at least one sensor device and provides an interactive and/or easy platform for the user to view results.

Optionally, the at least one processor may be further configured to:
for each region amongst the different regions of the body part, determine whether a temperature of said region exceeds a predefined temperature threshold value; and
generate a thermal map of the body part based on the differential temperature profile of the body part, wherein the thermal map indicates any region whose temperature exceeds the predefined temperature threshold value.

When the temperature of said region exceeds a predefined temperature threshold value, that may indicate that said region could potentially have an abnormality. The presence of the abnormality in the body part often results in additional heat generation through abnormal metabolic activity, therefore, a change in the temperature of the body part could mark the presence of potential abnormality in the body part. The temperature threshold value could lie in a range of 35 degrees Celsius to 38 degrees Celsius. The temperature threshold value lies in a range of 35 degrees Celsius to 36 degrees Celsius, 35 degrees Celsius to 37 degrees Celsius, 35 degrees Celsius to 38 degrees Celsius, 35.5 degrees Celsius to 36.5 degrees Celsius, 35.5 degrees Celsius to 37 degrees Celsius, 35.5 to 37.5 degrees Celsius, 36 degrees Celsius to 37 degrees Celsius, 36 degrees Celsius to 38 degrees Celsius, 36.5 degrees Celsius 37.5 degrees Celsius, 36.5 degrees Celsius to 38 degrees Celsius, or similar.

Further, the at least one processor generates a thermal map based upon the differential temperature profile of the body part. The thermal map is a representation of the temperature profile of different regions of the body part having temperature exceeding the predefined temperature threshold value. Optionally, the thermal map is at least one of: a colour map, a grayscale map, or the like. As an example, the thermal map may have three regions. Two regions out of the three regions may have a same colour, say for example, a blue colour, and a third region may have a yellow colour. Therefore, the region of the thermal map having the yellow colour may indicate an abnormality in the corresponding region of the body part. Advantageously, the technical benefit of this is that the results of the at least one sensor device that are generated in form of the thermal map, can be accurately read and understood by the user.

Optionally, the system may further comprise an infrared temperature measurement device configured to measure test temperatures of different regions of the body part, and wherein the at least one processor is further configured to receive the test temperatures of different regions of the body part from the infrared temperature measurement device. The infrared temperature measurement device is communicatively coupled to the at least one processor via the communication network. The test temperatures measured by the infrared temperature measurement device are received by the at least one processor via the communication network. The test temperatures are measured using infrared radiation (for example, radiation having a wavelength lying in a range of 0.7 micrometres to 1000 micrometres) in a contactless manner. The infrared radiation is not visible to the human eye. Operation of infrared temperature measurement devices is well known in the art.

Optionally, upon receiving the test temperatures of different regions of the body part from the infrared temperature measurement device, the at least one processor may be further configured to compare the test temperatures of different regions of the body part with the temperatures of different regions of the body part that are determined based on the pixel values, to validate the differential temperature profile of the body part. The infrared temperature measurement device could be implemented in combination with at least one camera or as a part of the at least one camera. The infrared temperature measurement device could be implemented as a measuring device capable of safely measuring temperature of the body part of the user with ease. For example, the infrared temperature measurement device may be a measuring device (such as an infrared thermometer) which can be used to measure the temperature in a contactless manner. The technical benefit of using the test temperatures is that the results obtained by the system using the at least one sensor device can be accurately validated by the infrared temperature measurement device, thereby increasing efficiency and authenticity of the results obtained by the system.

Optionally, the system may further comprise at least one user device communicably coupled to the at least one processor, wherein the at least one processor is further configured to send, to the at least one user device, at least one of: the temperatures of different regions of the body part, a thermal map of the body part, test temperatures of different regions of the body part, for presentation at the at least one user device. The at least one user device has a display. A user interface (UI) is rendered on the display in order to view results, such that the user can easily and accurately view the results. Examples of a given user device include, but are not limited to, a smartphone, a smartwatch, a tablet computer, a laptop computer, a desktop computer, an infotainment device, and a personal digital assistant.

The at least one processor is communicatively coupled to the at least one user device via the communication network. The at least one processor sends the temperatures of the different regions of the body part to the user device. At the UI rendered on the display of the at least one user device, the temperatures of the different regions of the body part may be visually represented in form of one or more of a table, a histogram, a schematic, a heat map, and the like. Further, the at least one processor sends the thermal map and the test temperatures measured by the infrared temperature measurement device to the user device.

Optionally, the at least one user device may comprise the at least one camera. For example, a mobile phone equipped with camera can be used to read the temperatures of different regions of the body part by taking a picture of the first side of the base of the at least one sensor device using the camera as well as view the temperatures determined upon processing. A processor of the at least one user device may be employed for the processing. In such a case, a single user device may be beneficially used for implementing the camera as well as the at least one processor of the system. The technical advantage of this is that the results obtained by the system can be efficiently captured and viewed using the at least one user device making the system of the present disclosure very compact and easy to use.

Optionally, the at least one sensor device may comprise a unique identifier provided on the base, the at least one camera being configured to capture at least one identification image representing the unique identifier, wherein the at least processor may be configured to:
identify the unique identifier in the at least one identification image;
associate the temperatures of different regions of the body part that are determined to the unique identifier; and
perform one of: block future interpretation of sensed temperatures from the at least one sensor device, permit future interpretation of sensed temperatures from the at least one sensor device upon successful reuse actions being implemented.

The at least one unique identifier could be provided on either the first side or the second side of the base. As an example, the unique identifier may be provided on the first side of the base. The unique identifier could be any of a quick response (QR) code, an alphanumeric code, a Radio Frequency Identification (RFID) marker, a noise pattern, and the like. The at least one camera captures the at least one identification image. The at least one identification image includes the unique identifier. The at least one identification image is received by the at least one processor via the communication network. The at least one processor identifies the at least one identification image using at least one feature recognition algorithm. Such feature recognition algorithms are well-known in the art.

Further, the at least one processor associates the unique identifier on the at least one sensor device with the temperatures of different regions of the breast determined using the at least one sensor device. Optionally, the at least one processor also records uses of the unique identifier which is essential to determine uses of the at least one sensor device. Optionally, the unique identifier could also be used to ensure a condition of the at least one sensor device prior to use. For example, the unique identifier may be used to ensure the at least one device is working properly.

Furthermore, the at least one processor may be configured to block future interpretation of the sensed temperatures from the at least one sensor device. The at least one processor can only interpret temperatures of different regions of the body part which are associated with the unique identifier. Further, the at least one processor permits future interpretation of sensed temperatures from the at least one sensor device upon successful reuse actions being performed. The reuse actions could be performed by the user, for example, after a reuse action of replacing the unique identifier with another one. The at least one processor may associate the sensed data with a new unique identifier and allow interpretation of the temperatures. Optionally, the at least one processor may allow reuse of the at least one sensor device only after a reuse action of a predefined check or confirmations is/are made by the user. In this regard, the at least one processor could prompt the user to provide any input value in order to enable another use of the at least one sensor device. As an example, the input value may be an ambient temperature of a place where the at least one sensor is being used. In case, the ambient temperature is less than 25 degrees Celsius, reuse may be permitted owing to fewer chances of sweating. As another example, the input value may be the sensed temperature of a previous test. In case, the previous temperatures of any region of the body part are not above the temperature threshold value, the reuse may be allowed, as the at least one temperature-sensitive element will not impact the accuracy of future detection of temperatures. The technical benefit of this is that the unique identifier on the at least one sensor device is associated with the measurement obtained using the at least one sensor device. The association of the measurements with the unique identifier prevents future interpretation using the at least one sensor device, thereby solving the problem of multiple uses of conventional systems. The multiple uses might decrease the accuracy of the results. Therefore, the at least one sensor device is restricted to single-use only until certain checks are provided by the user. Owing to the above, the accuracy of the system is significantly improved.

Optionally, the at least one processor is further configured to:
process the at least one identification image to determine at least one usage parameter, wherein the at least one usage parameter comprises at least one of: the body part for which the at least one sensor device is used, a number of times the at least one sensor device has been used, a remaining number of times of future use of the at least one sensor device, a total number of times the at least one sensor device can be used, a predefined temperature range for storing the at least one sensor device when not in use, a time period within which the at least one sensor device can be used; and
associate the at least one usage parameter to the unique identifier.

In this regard, the at least one identification image is used to know details regarding the at least one sensor device prior to usage. A technical effect of determining the at least one usage parameter is that a detailed information about the at least one sensor device is provided to the user, so that the user can make an informed decision regarding usage of the at least one sensor device. The at least one identification image is processed in real time or in near-real time. Herein, the at least one sensor device may be designed based on the body part with which said at least one sensor device is to be in contact. Hence, the at least one identification image is processed to extract information regarding the body part for which the at least one sensor device is to be used for, and provide said information to the user regarding the body part in a form of a text, a code, a number, an alphanumeric text, or an image.

Optionally, upon processing the at least one identification image, a historical data is provided to the user in the form of the number of times the at least one sensor device has been used, and the total number of times the at least one sensor device can be used. Herein, the number of times, the remaining number of times, and the total number of times are finite numeric values, wherein the total number of times is used as a reference value. The remaining number of times is a difference between the number of times the at least one sensor device has been used and the total number of times the at least one sensor device can be used. For example, the total number of times the at least one sensor device may be used for is 4 times. The number of times the at least one sensor device has already been used for may be 3 times. Hence, the remaining number of times for the future use of the at least one sensor device is 1 time.

Additionally, optionally, upon processing the at least one identification image, the predefined temperature range for storing the at least one sensor device is provided to the information, when not in use, such that a performance of the at least one sensor device does not deteriorate. The predefined temperature range could lie in a range of 4 degrees Celsius to 12 degrees Celsius. The predefined temperature range lies in a range of 4 degrees Celsius to 6 degrees Celsius, 4 degrees Celsius to 8 degrees Celsius, 4 degrees Celsius to 10 degrees Celsius, 4 degrees Celsius to 12 degrees Celsius, 6 degrees Celsius to 8 degrees Celsius, 6 degrees Celsius to 10 degrees Celsius, 6 degrees Celsius to 12 degrees Celsius, 8 degrees Celsius to 10 degrees Celsius, 8 degrees Celsius to 12 degrees Celsius, 10 degrees Celsius to 12 degrees Celsius, or similar. Additionally, the term “time period” refers to a period of time after which the at least one sensor device will get expired, i.e., an accuracy with which the at least one sensor device determines the differential temperature of the body part, reduces. Herein, the time period may be a month, 2 months, 4 months, 8 months, 12 months, 24 months, and similar.

Optionally, the at least one processor associates the at least one usage parameter by way of connecting or linking to the unique identifier, wherein the at least one processor correlated the at least one usage parameter to a corresponding unique identifier. Optionally, the at least one processor is further configured to send, to the at least one user device, the at least one usage parameter, for presentation on the at least one user device.

Optionally, the at least one camera may be configured to capture at least one body image representing at least the body part of the user, wherein the at least one processor is further configured to:
identify features of the body part that are represented in the at least one body image;
digitally superimpose at least one virtual object on the at least one body image for enabling correct placement of the at least one sensor device on the body part; and
send the at least one body image having the at least one virtual object superimposed thereon to a user device for display thereat.

Optionally, the user device may be associated with the user. Examples of the user device include, but are not limited to, a mobile phone, a computer, a laptop, and a smartwatch. The features could be edges, shapes, sizes, colours of different regions, or the like. Optionally, the at least one processor is configured to identify the features of the body part that are represented in the at least one body image using at least one feature detection algorithm. Optionally, a given feature detection algorithm may be at least one of: an edge-detection algorithm, a corner-detection algorithm, a blob-detection algorithm, a feature descriptor algorithm. Further, the at least one processor is configured to digitally superimpose the virtual object on the different regions of the at least one body image, as required. The virtual object is a computer-generated object used to indicate different features of the body part that are represented in the at least one body image. The virtual object could be a geometric shape, an arrow, a pattern, or similar, that enables correct placement of the at least one sensor device on the body part. As an example, the virtual object may be the arrow pointing towards the nipple region of the body part. As another example, the virtual object may be a circle encircling an outer region of the body part. The at least one body image superimposed with the virtual object is an augmented reality image. Further, the at least one processor sends the at least one augmented reality image to the user device in order to be displayed to the user. The technical benefit of this is that the augmented reality image is utilized to provide accurate positioning of the at least one sensor device on the body part of the user thereby improving the efficiency and accuracy of the results.

Optionally, the at least one sensor device may comprise a first sensor device and a second sensor device and the body part comprises a first body part and a second body part, wherein the first sensor device may be used for determining first temperatures of different regions of the first body part of the user and the second sensor device may be used for determining second temperatures of different regions of the second body part of the user, and wherein the at least one processor may be configured to: for a given region of the body part, compare its corresponding first temperature and second temperature to determine a temperature difference therebetween.

Optionally, the at least one processor may be further configured to determine whether the temperature difference exceeds a predefined difference. Optionally, when for the given region of the body part, the temperature difference exceeds the predefined difference, the at least one processor may be configured to indicate a possibility of an abnormality being present. The predefined difference could lie in a range of 0.5 to 2.5 degrees Celsius. The predefined difference could lie in a range of 0.5 to 1 degrees Celsius, 0.5 to 1.5 degrees Celsius, 0.5 to 2 degrees Celsius, 0.75 to 1.5 degrees Celsius, 0.75 to 2 degrees Celsius, 0.75 to 2.5 degrees Celsius, 1 to 1.5 degrees Celsius, 1 to 2 degrees Celsius, 1.5 to 2 degrees Celsius, 1.5 to 2.5 degrees Celsius or similar. In an example, the temperature difference between two regions of each of the body part may be equal to or greater than 2 degrees Celsius, which may relate to a condition of abnormality in the body part. The technical benefit of this is that the at least one sensor device is able to detect the abnormality accurately.

A second aspect of the present disclosure provides a sensor device comprising:
a base having a first side and a second side opposite to the first side; and
at least one temperature-sensitive element provided on the first side of the base, wherein the temperature-sensitive element is arranged to sense temperatures of different regions of a body part of a user upon contact with the body part of the user for a predefined time period by changing its colour proportionally to the temperature of each region of the body part, thereby creating a temperature map of the body part.

Therefore, when the sensor device is in use, the first side is in contact with the body part of a user for a predefined time period and the at least one temperature-sensitive element senses temperatures of different regions of the body part.

In this regard, various embodiments and variants disclosed above, with respect to the aforementioned first aspect, apply mutatis mutandis to the second aspect.

The base could be made of a fabric that is flexible in nature. The fabric could be selected from a material, that offers, softness, flexibility, prevent rashes and other properties which makes the at least one base comfortable to wear for the user. The material could be selected from, cotton, polymer, polyester, or the like. Additionally, the base could be provided with any one of: a foam backing, a silicon mould in order to provide additional comfort to the user. Optionally, the base is made of foil pads having a soft flexible foam backing. Alternatively, optionally, the base is made of the temperature-sensitive material. The base may be made of a material that does not wrinkle or bundle up or fold when the sensor device is worn by the user. Yet alternatively, optionally, the base may be made of a polymer substrate material. The polymer substrate material could be selected in such a manner that said polymer substrate material offers at least one of: flexibility, thermal stability, chemical resistance. Examples of such polymer substrate material may include, but are not limited to, Biaxially Oriented Polypropylene (BOPP), Polyethylene Terephthalate (PET), polyethylene (PE), polyvinyl chloride (PVC), and Polyimide (PI). Optionally, a shape of the base may be designed based on the body part with which the sensor device is to be in contact with. Examples of such shapes of the base may be, a circle, a rectangle, a triangle, a square, and similar. In an instance, when the body part is one of: the at least one breast, the at least one testicle, the base is thin, may have a low threshold of pressure, and may be in a form of the circle. In another instance, when the body part is one of: the at least one portion of skin, the at least one foot, the base is a rectangle. However, when the body part is at least one foot, the base may be of a predefined thickness, wherein said predefined thickness is greater than a thickness for the base when the body part is at least one breast. For example, when the body part is at least one foot, the base of the sensor device for a back, a front, a middle opposite side may be thicker than the sensor device used for when the body part is at least one breast. This may facilitate for the base to resist pressure of the at least one foot, and thus enables accurate sensing of temperature of different regions of the at least one foot.

The base has the first side and the second side. The second side is opposite to the first side. When the sensor device is worn by the user, the first side is in contact with the user's body part whereas the second side is not in contact with the user's body part. Furthermore, the base has the hole in order to wear the at least one sensor device appropriately on the body part. The hole could be in the middle of the base, on towards a given side of the base, on a corner of the base, and the like. Optionally, the sensor device may further comprise at least two slits, wherein the sensor device is in a shape of a circular disc. A diameter of the circular disc may be 14 centimetres, and a radius of the hole 902 may be 2 centimetres. The at least two slits are cut from a perimeter edge of the circular disc, and the at least two slits are at a predefined distance away from the hole. In an instance, the at least two slits are in a shape of an acute triangle. For example, an interior angle of the acute triangle may be 41 degrees, and two exterior angles opposite to each other may be 91 degrees and 94 degrees, respectively. In another instance, the at least two slits are in a shape of a rectangle, wherein the at least two slits are diametrically opposite to each other. For example, a length and a width of the at least two slits may be 5 centimetres and 0.7 centimetres, respectively.

Optionally, the base does not cover the entire body part. Herein, the temperatures of different regions of the body part are integrated by the at least one sensor for the entire body part. Optionally, the base may comprise at least one peelable element to remove a protective sheet present on the first side of the base. The at least one temperature-sensitive element is provided surrounding the hole on the first side of the at least one sensor device. The first side, having the at least one temperature-sensitive element stays in contact with the body part of the user, when in use. The at least one temperature-sensitive element could be implemented as one large temperature-sensitive element or a plurality of small temperature-sensitive elements. As an example, the at least one sensor device has three temperature-sensitive elements present on the first side of the base. Each of the three temperature-sensitive elements includes a plurality of columns (for example, 18 columns numbered from 1 to 18). Each column may indicate a half-degree increase in temperature from approximately 32 degrees Celsius to 37 degrees Celsius. The at least one temperature-sensitive element senses the temperatures of the different regions of the body part.

Optionally, the at least one temperature-sensitive element may be implemented as at least one of: a coating of a temperature-sensitive material on the first side, an element having wells in which a temperature-sensitive material is filled, a thermally-conductive covering in which a temperature-sensitive material is filled. In a first implementation, an entire surface of the at least one temperature-sensitive element is coated with the temperature-sensitive material, i.e., a sheet of a colour changing material. In a second implementation, the entire surface of the at least one temperature-sensitive element includes a plurality of wells. The plurality of wells could be arranged randomly or according to a specific pattern and be filled with the temperature-sensitive material. Alternatively, the plurality of columns (as described above) of each of the three temperature-sensitive elements could have the plurality of wells arranged in a column-wise manner and the plurality of wells could be filled with the temperature-sensitive material. In a third implementation, a plurality of pouch like structures filled temperature-sensitive material could be provided on the at least one temperature-sensitive element. The plurality of pouch like structures could be arranged on the entire surface of the at least one temperature-sensitive element such that the plurality of pouch like structures remain in adequate contact with the body part of the user and are able to detect temperatures of different regions of the body part. A technical benefit of this is that the at least one temperature-sensitive element efficiently senses the temperatures of different regions of the body part providing accurate results to the user.

Optionally, the sensor device may be surface printed, wherein the coating of the at least one temperature-sensitive material may be transferred through a mesh screen onto the first side of the base of the sensor device. In this regard, a design may be cut out of a thin, strong material and then the coating of the temperature-sensitive material is forced through the mesh screen onto the first side of the base using a scraping tool. Herein, the coating of the temperature-sensitive material is forced by any one of: rubbing, rolling, spraying onto the first side. A technical effect of surface printing the sensor device is that the at least one temperature-sensitive element is manufactured in an intricate and in a precise manner wherein fine details are reproduced accurately. Another technical effect of surface printing the sensor device is that said surface printing offers flexibility to print various shapes, patterns of the at least one temperature-sensitive material on the first side, thereby making the surface printing ideal for customization.

Optionally, the at least one temperature-sensitive material may be at least one of: a thermosensitive fluid, a thermosensitive compound. Herein, a calibration of the at least one temperature-sensitive element is useful between a temperature range of 20 degrees Celsius to 40 degrees Celsius. The thermosensitive fluid is at least one of: a liquid substance, a gel substance, that undergo a change in at least one physical property based on different temperatures of different regions of the at least one body part. The thermosensitive fluid could be, but not limited to, a thermosensitive ink, a thermosensitive pigment. The thermosensitive compound is at least one of: a liquid substance, a gel substance, a powder substance, a solid substance, that undergoes changes in physical properties in a similar manner as the thermosensitive fluid. The thermosensitive compound could be, but not limited to, a crystalline organic compound, encapsulated cholesteric liquid crystals, N-phenylbenzylamine, Bromo-p-Xylene, Tetradecanol, a thermochromic compound.

Optionally, the liquid crystal material comprises liquid crystal molecules, wherein an alignment of the liquid crystal molecules of the liquid crystal material changes based on different temperatures of different regions of the at least one body part. Liquid crystal materials which are highly sensitive to temperature changes are suitable for use in the present invention. For example, the liquid crystal material may react to 0.1 degree Celsius change in temperature. Hence, based on a type of the liquid crystal material selected, the differential temperature profile for the body part is clearly formed. For example, the liquid crystal material may react to a difference in temperature caused by a tumour even when said tumour may not be directly adjacent to the at least one portion of the skin. Optionally, the liquid crystal material may be formulated to react to temperatures lying in a range of −30 degrees Celsius to +120 degrees Celsius. The temperatures may lie in a range of −30 degrees Celsius to 0 degrees Celsius, −30 degrees Celsius to 30 degrees Celsius, −30 degrees Celsius to +60 degrees Celsius, −30 degrees Celsius to +90 degrees Celsius, −30 degrees Celsius to +120 degrees Celsius, 0 degrees Celsius to +30 degrees Celsius, 0 degrees Celsius to +60 degrees Celsius, 0 degrees Celsius to +90 degrees Celsius, 0 degrees Celsius to +120 degrees Celsius, +30 degrees Celsius to +60 degrees Celsius, +30 degrees Celsius to +90 degrees Celsius, +30 degrees Celsius to +1200 degrees Celsius, +60 degrees Celsius to +90 degrees Celsius, +60 degrees Celsius to +120 degrees Celsius, +90 degrees Celsius to +120 degrees Celsius, or similar.

Optionally, a range of thermal sensitivity of the at least one temperature-sensitive material may affect the operating temperature range of the sensor device. The operating temperature could lie in the operating temperature range of 29 degrees Celsius to 41 degrees Celsius. The operating temperature could lie in the operating temperature range of 29 degrees Celsius to 31 degrees Celsius, 29 degrees Celsius to 33 degrees Celsius, 29 degrees Celsius to 35 degrees Celsius, 29 degrees Celsius to 41 degrees Celsius, 30 degrees Celsius to 32 degrees Celsius, 30 degrees Celsius to 36 degrees Celsius, 30 degrees Celsius to 41 degrees Celsius, 34 degrees Celsius to 38 degrees Celsius, 34 degrees Celsius to 40 degrees Celsius, 35 degrees Celsius to 41 degrees Celsius, or similar. Optionally, in order to achieve a predefined efficiency of the at least one temperature sensitive material, the user may be instructed to store the at least one sensor device in one of: a refrigerator, at a room temperature below 26 degrees Celsius away from any source of heat including heat generating lights to achieve optimum performance by the temperature-sensitive material.

Optionally, an additive may be added to the at least one temperature-sensitive element such that the change in at least one of: the electrical property, the physical property, the chemical property changes in response to the change in the temperature of the body part is not changed for another predefined time period. This may ensure that sufficient number of images are captured by the at least one camera, and then sent to the at least one processor for processing the at least one image. The another predefined time period may be up to 20 minutes, 15, 10, or 5 minutes.

Alternatively, an additive may be added to a sensor device comprising temperature-sensitive material comprising a thermochromic ink to stabilise the colours representing the temperatures sensed, when the user applies the sensor device to a body part for a predefined period. In such a case, the predefined time period may be up to 20 minutes. As an example, the another predefined time period may be, 15 or 12 or 11 or 10 or 7 or 5 or 2 minutes. The another predefined time period may be 1 minute, although this is not preferable since it may affect the accuracy of the results.

Optionally, when the body part is at least one breast, the base of the sensor device has a hole, and the at least one temperature-sensitive element at least partially surrounds the hole.

Optionally, the contact between the first side and the body part is made using at least one of: an adhesive, a supporting element, a tying means, a clamping means.

It will be appreciated that the at least one sensor device is to be used according to certain instructions, for enabling accurate measurements. These instructions relate to removal of packaging of the at least one sensor device, a required operating temperature of the at least one sensor device, a manner in which the at least one sensor device is to be placed on the body part, how to use the at least one sensor device with different types of bras, how to hold the at least one sensor device when in use, body posture of the user when using the at least one sensor device, how to remove the at least one sensor device after the use, and the like.

Alternatively, optionally, a thickness of the base may be selected in such a manner such that, when the sensor device is in use, the first side of the sensor device may adhere to the at least one body part by friction and/or gravity.

Optionally, the sensor device may further comprise a protective layer on the first side to protect the temperature-sensitive material. This protective layer is used when the temperature-sensitive material is already dispersed in a composite ink which provides protection from the adhesive. Alternatively, optionally, the adhesive may be used as the protective layer.

A third aspect of the present disclosure provides a method for determining the differential temperature in a body part, the method comprising:
capturing at least one image of a first side of at least one sensor device, wherein the at least one image is captured within a given time period from a time of removal of contact between the first side and the body part;
identifying, in the at least one image, at least one image segment representing at least one temperature-sensitive element of the at least one sensor device;
recognizing pixel values of pixels in the at least one image segment; and
determining the temperatures of different regions of the body part, based on the pixel values of pixels in the at least one image segment, wherein the temperatures of different regions of the body part form a differential temperature profile of the body part.

In this regard, various embodiments and variants disclosed above, with respect to the aforementioned first aspect and the second aspect, apply mutatis mutandis to the third aspect.

The method steps for determining the differential temperature in a body part are described above. Advantageously, the aforesaid method is easy to implement, provides fast indicative results, and does not require complex or expensive equipment. Furthermore, there is no complexity of use or harmful effect for any user applying the method described above or any of the variations of the method described in other part of the text. In all embodiments, the at least one software application is preferably implemented as application for a mobile device such as a smartphone. It will be appreciated that a smartphone is understood as a mobile telephone device capable of a plurality of functions including but not limited to camera capabilities, software functions, computer functions, network connectives.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

Experimental Data from Clinical Studies

The sensor device was subjected to clinical trials on patients, specifically women, of different age groups having varying clinical conditions, when the body part was the at least one breast. The clinical trials were performed all over the world and have demonstrated that the sensor device is safe, easy to use, and that no adverse and side effects were observed during the trials.

The effectiveness of the present invention in detecting anomalies such as cancer in the at least one breast was established by validating the results of the standard procedures of determining breast cancer with results obtained using the system employing the sensor device. The sensor device of the present invention correctly identified anomalies for 93 of the 112 women with a positive biopsy result, giving a sensitivity of 83%.

Further, the present invention was tested for sensitivity to different sizes of cancer cells and the results shown indicated a high level of sensitivity. The results are shown in table 1.

TABLE 1

| | Sensor device of the |
|---|---|
| Size of cancer (cm) | present invention |
| Less than 1.0 | 87.5% (7 out of 8) |
| 1.0-1.9 | 71.4% (15 out of 21) |
| Sub-total | 75.9% (22 out of 29) |
| 2.0-2.9 | 71.9% (23 out of 32) |
| 3.0 or larger | 91.7% (33 out of 36) |
| Sub-total | 82.4% (56 out of 68) |
| Total | 80.4% (78 out of 97) |

Further, the present invention was tested for women of different age groups. Age specific results for the sensitivity of the present invention to anomaly detection when anomaly is present are shown below in Table 2. Here ‘+’ and ‘−’ indicate presence and absence of an anomaly, respectively, and the numbers in 3^{rd} and 4^{th} columns indicate numbers of women.

TABLE 2

| | | Sensor device of the | | | |
|---|---|---|---|---|---|
| Age | Clinical | present invention | | | |
| group | Screening | + | − | Total | Sensitivity |
| <25 | + | 1 | 0 | 1 | 100% |
| | − | 3 | 6 | 9 | |
| 25-35 | + | 5 | 0 | 5 | 100% |
| | − | 4 | 6 | 10 | |
| 35-44 | + | 12 | 1 | 13 | 92.3% |
| | − | 5 | 10 | 15 | |
| 45-54 | + | 15 | 4 | 19 | 78.8% |
| | − | 9 | 9 | 18 | |
| 55-64 | + | 36 | 10 | 46 | 76.8% |
| | − | 7 | 3 | 10 | |
| >65 | + | 24 | 4 | 28 | 81.8% |
| | − | 3 | 2 | 5 | |

Furthermore, the present invention was tested for specificity on another group of women of different age groups. Specificity is calculated as how often the system of the present invention produces a negative result in women with no detected significant breast abnormality. Age specific results for specificity of the present invention are shown below in Table 3. Here ‘+’ and ‘−’ indicate presence and absence of an anomaly, respectively, and the numbers in 3^{rd} and 4^{th} columns indicate numbers of women.

TABLE 3

| | | Sensor device | | | | |
|---|---|---|---|---|---|---|
| | | of the present | | | | |
| Age | Clinical | invention | | | | |
| group | Screening | + | − | Total | Sensitivity | Specificity |
| <25 | + | 2 | 0 | 2 | 100% | 100% |
| | − | 0 | 14 | 14 | | |
| 25-35 | + | 10 | 1 | 11 | 90.9% | 83.6% |
| | − | 28 | 143 | 171 | | |
| 35-44 | + | 27 | 5 | 32 | 84.4% | 87.8% |
| | − | 55 | 396 | 451 | | |
| 45-54 | + | 26 | 5 | 31 | 83.9% | 87.8% |
| | − | 102 | 732 | 834 | | |
| 55-64 | + | 16 | 1 | 17 | 94.1% | 84.9 |
| | − | 36 | 865 | 901 | | |
| >65 | + | 5 | 1 | 6 | 83.3% | 86.5 |
| | − | 45 | 290 | 335 | | |

| | | |
|---|---|---|
| Overall | 86.9% | 86.5% |

Based upon the above data, it was observed that the present invention worked effectively in detecting positive abnormality and negative abnormality in the at least one breast without any harmful side effects to the patient.

### BRIEF DESCRIPTION OF THE DRAWINGS

One or more embodiments of the invention will now be described, by way of example only, with reference to the following diagrams wherein:

FIGS. 1A, 1B, 1C and 1D are block diagrams representing a system for determining differential temperature in a body part, in accordance with various embodiments of the present disclosure;

FIG. 2 is a schematic illustration of at least one sensor device when a body part is at least one breast, in accordance with an embodiment of the present disclosure;

FIG. 3 is a schematic illustration of at least one sensor device when a body part is at least one breast, in accordance with another embodiment of the present disclosure;

FIG. 4 is a schematic illustration of at least one sensor device when a body part is at least one breast, in accordance with yet another embodiment of the present disclosure;

FIG. 5 is a schematic illustration of at least one sensor device having a unique identifier when a body part is at least one breast, in accordance with still another embodiment of the present disclosure;

FIG. 6 is another schematic illustration of the sensor device 200 of FIG. 2, when a body part is at least one breast, in accordance with an embodiment of the present disclosure;

FIGS. 7A and 7B are schematic illustrations of at least one sensor device, in accordance with an embodiment of the present disclosure;

FIG. 8 is an exemplary implementation of at least one sensor device for determining differential temperature in a body part, in accordance with an embodiment of the present disclosure;

FIGS. 9A and 9B are schematic illustrations of different designs of at least one sensor device, in accordance with different embodiments of the present disclosure; and

FIG. 10 is an illustration of a flowchart depicting steps of a method for determining differential temperature in a body part, in accordance with an embodiment of the present disclosure.

### DETAILED DESCRIPTION

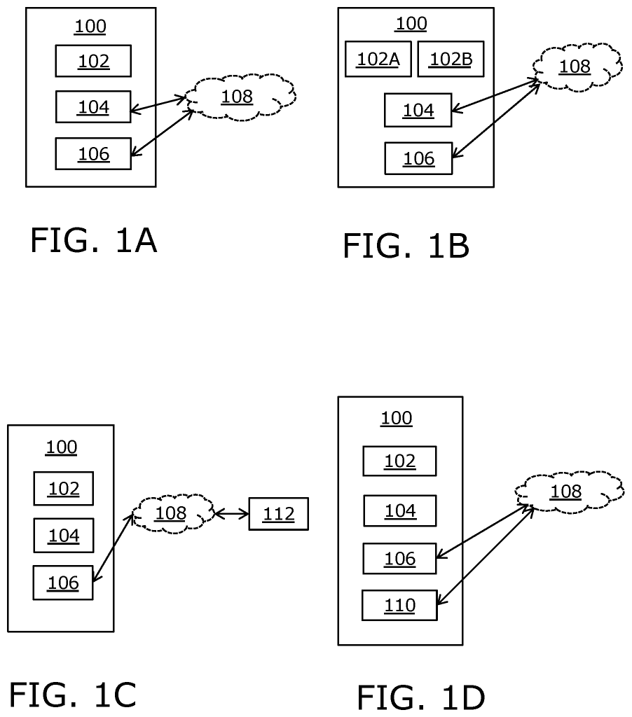

Referring to FIGS. 1A, 1B, 1C and 1D, there are shown block diagrams representing a system 100 for determining differential temperature in a body part, in accordance with various embodiments of the present disclosure. In FIGS. 1A-1D, the system 100 comprises at least one sensor device (depicted as a sensor device 102), at least one camera (depicted as a camera 104), and at least one processor (depicted as a processor 106). The processor 106 is communicatively coupled to the camera 104 via a communication network 108. In FIG. 1B, the system 100 is shown to comprise a first sensor device 102A and a second sensor device 102B corresponding to a first body part and a second body part. When the system 100 is in use, the first sensor device 102A and the second sensor device 102B are used for determining first temperatures of different regions of the first body part of the user and for determining second temperatures of different regions of a second body part of the user respectively. In FIG. 1C, the system 100 further comprises at least one user device (depicted as a user device 112) communicably coupled to the processor 106 via the communication network 108. In FIG. 1D, the system 100 further comprises an infrared temperature measurement device 110 communicatively coupled to the processor 106 via the communication network 108. The infrared temperature measurement device 110 could be implemented in combination with the camera 104 or as a part of the camera 104.

FIGS. 1A, 1B, 1C and 1D are merely examples, which should not unduly limit the scope of the claims herein. A person skilled in the art will recognize many variations, alternatives, and modifications of embodiments of the present disclosure.

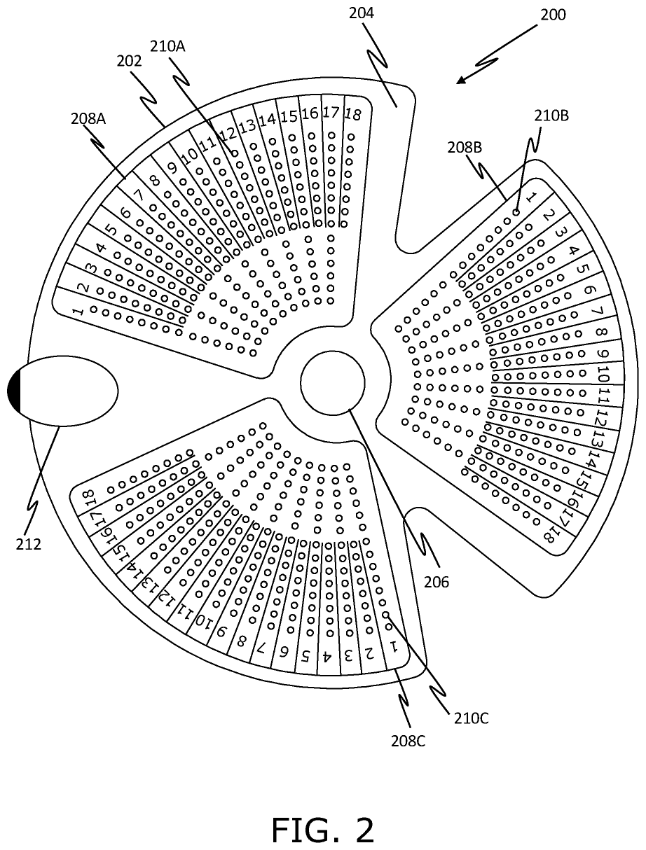

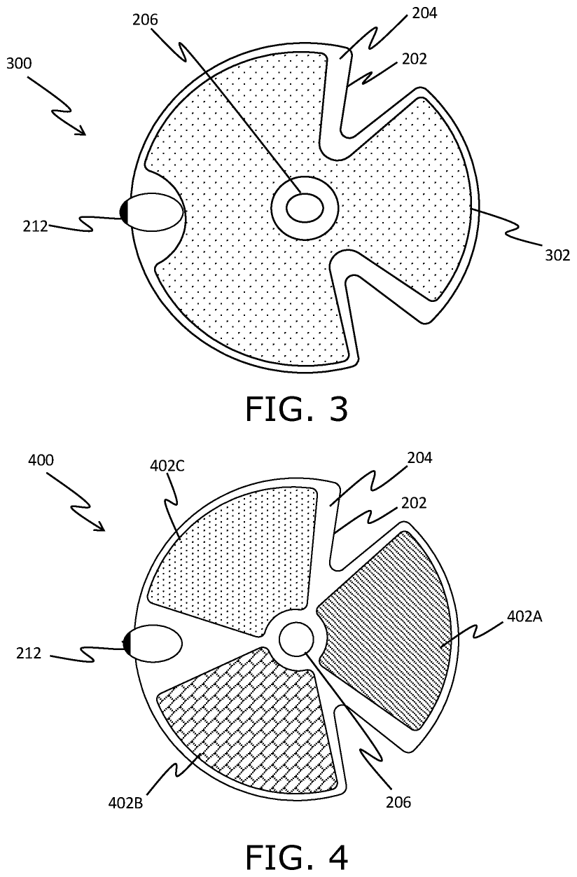

Referring to FIG. 2, there is shown a schematic illustration of at least one sensor device (depicted as a sensor device 200) when a body part is at least one breast, in accordance with an embodiment of the present disclosure. The sensor device 200 comprises a base 202 having a first side 204, a second side (not shown) opposite to the first side 204, a hole 206 and three temperature-sensitive elements 208A, 208B and 208C surrounding the hole 206. The three temperature-sensitive elements 208A-C are implemented as elements having a plurality of wells (depicted as wells 210A, 210B, and 210C) in which a temperature-sensitive material is filled. Each of the three temperature-sensitive elements 208A-C includes a plurality of columns, numbered from 1-18. Each of the columns includes multiple wells from amongst the plurality of wells 210A-C. Further, the base 202 comprises a peelable element 212. The elements of the sensor device 200 that are common to sensor devices of FIGS. 3, 4, 5, and 6, have been referred to with same numbering as in FIG. 2, for sake of simplicity.

Referring to FIG. 3, there is shown a schematic illustration of at least one sensor device (depicted as a sensor device 300) when a body part is at least one breast, in accordance with another embodiment of the present disclosure. The sensor device 300 comprises at least one temperature-sensitive element (depicted as a temperature-sensitive element 302) implemented as a coating on the first side 204.

Referring to FIG. 4, there is shown a schematic illustration of at least one sensor device (depicted as a sensor device 400) when a body part is at least one breast, in accordance with yet another embodiment of the present disclosure. The sensor device 400 comprises three temperature-sensitive elements 402A, 402B and 402C implemented as coatings on the first side 204.

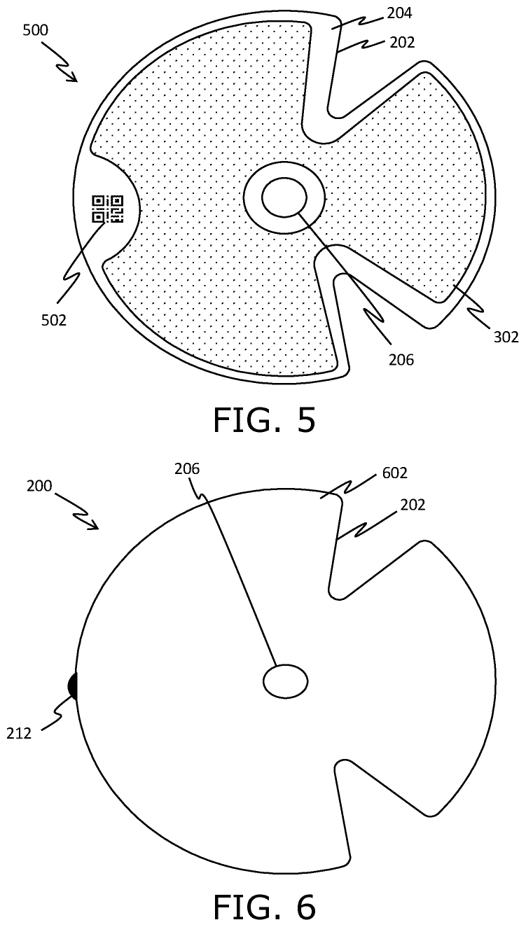

Referring to FIG. 5, there is shown a schematic illustration of at least one sensor device (depicted as a sensor device 500) when a body part is at least one breast, in accordance with still another embodiment of the present disclosure. The sensor device 500 comprises a unique identifier 502 provided on the first side 204 of the base 202.

Referring to FIG. 6, there is shown another schematic illustration of the sensor device 200 of FIG. 2, when a body part is at least one breast, in accordance with an embodiment of the present disclosure. As mentioned in FIG. 2, the sensor device 200 comprises the base 202 having the first side (not shown), a second side 602 opposite to the first side 204, and the hole 206. Further, a protruded portion of the peelable element 212 is visible from the second side 602 of the base 202.

FIG. 2, FIG. 3, FIG. 4, FIG. 5, and FIG. 6 are merely examples, which should not unduly limit the scope of the claims herein. A person skilled in the art will recognize many variations, alternatives, and modifications of embodiments of the present disclosure.

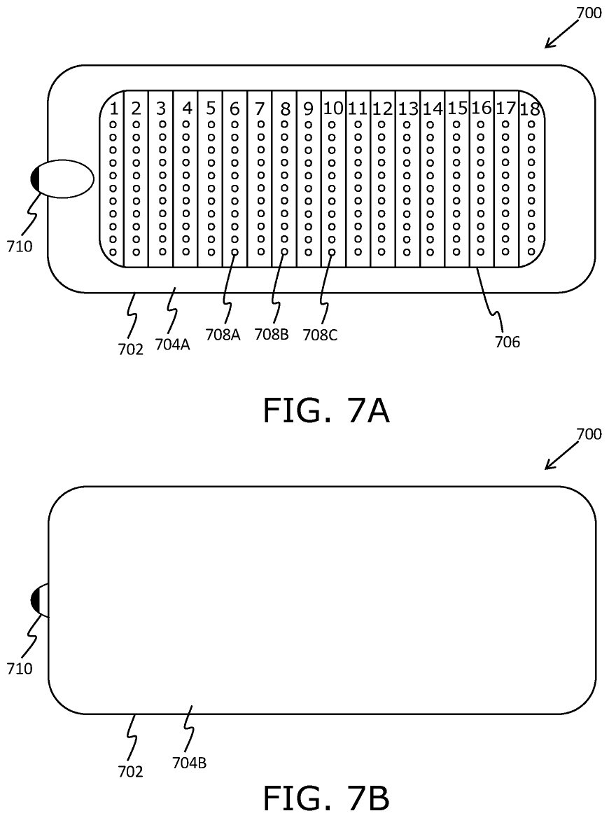

Referring to FIGS. 7A and 7B, there are shown schematic illustrations of at least one sensor device (depicted as a sensor device 700), in accordance with an embodiment of the present disclosure. In FIG. 7A, the sensor device 700 comprises a base 702 having a first side 704A, and a temperature-sensitive element 706. The temperature-sensitive element 706 is implemented as an element having a plurality of wells (depicted as wells 708A, 708B, and 708C) in which a temperature-sensitive material is filled. The temperature-sensitive element 706 includes a plurality of columns, numbered from 1-18. Each of the columns includes multiple wells from amongst the plurality of wells 708A-C. Optionally, the base 702 comprises a peelable element 710. In FIG. 7B, the base 702 of the sensor device 700 comprises the second side 704B opposite to the first side 704A, and optionally, a protruded portion of the peelable element 710 is visible from the second side 704B of the base 702.

FIGS. 7A and 7B are merely examples, which should not unduly limit the scope of the claims herein. A person skilled in the art will recognize many variations, alternatives, and modifications of embodiments of the present disclosure.

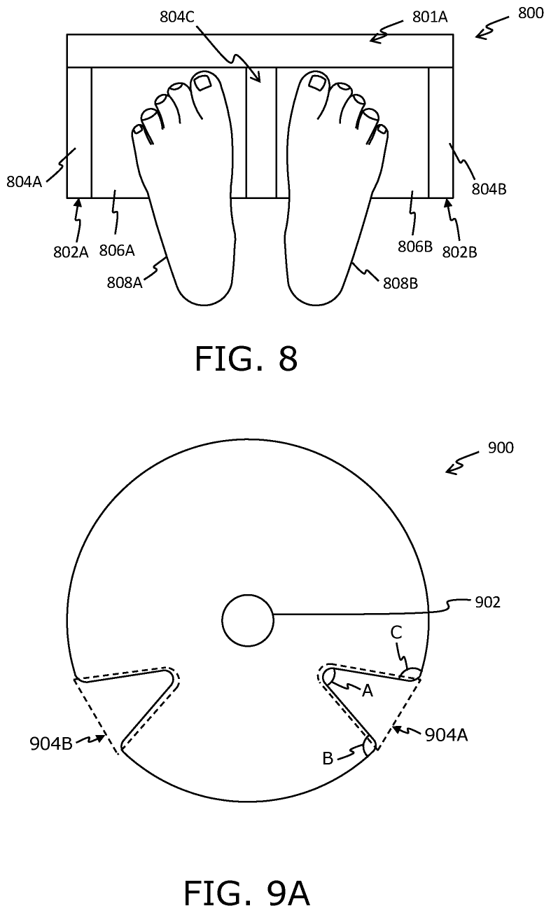

Referring to FIG. 8, there is shown an exemplary implementation of at least one sensor device 800 for determining differential temperature in a body part, in accordance with an embodiment of the present disclosure. The at least one sensor device 800 comprises a first sensor device 802A and a second sensor device 802B. The sensor device 800 comprises a portion 801A and a portion 801B (not shown) opposite to the portion 801A portion, thus located in a region closes to the heels of the feet shown and a portion 804C. The first sensor device 802A comprises a base 804A having a first side 806A and a second side (not shown) opposite to the first side 806A, and a temperature-sensitive element on the first side 806A. The second sensor device 802B comprises another base 804B having another first side 806B and another second side (not shown) opposite to the another first side 806B, and another temperature-sensitive element on the another first side 806B. The body part comprises a first body part (depicted, for example, as a left foot 808A) and a second body part (depicted, for example, as a right foot 808B). When the first sensor device 802A and the second sensor device 802B are in use, the first side 806A of the first sensor device 802A is in contact with the left foot 808A and the another first side 806B of the second sensor device 802B is in contact with the right foot 808B. The first sensor device 802A is used for determining first temperatures of different regions of the left foot 808A and the second sensor device 802B is used for determining second temperatures of different regions of the right foot 808B.

FIG. 8 is merely an example, which should not unduly limit the scope of the claims herein. For example, in FIG. 8, one or more of the portions 801A, 801B, 804C may be protruded portions. Similarly, the sides 804A, 804b, 804C may be protruded sides. Protruded portions and/or the sides act as guides for each foot to ensure that each foot is located in an approximately optimum region for the use of the sensor device 800.

A person skilled in the art will recognize many variations, alternatives, and modifications of embodiments of the present disclosure. For example, the body part could be any other body part such as a left palm and a right palm, a left testicle and a right testicle, and similar.

Referring to FIGS. 9A and 9B, there are shown schematic illustrations of different designs of sensor device 900, in accordance with different embodiments of the present disclosure. In FIGS. 9A and 9B, the sensor device 900 is a circular disc comprising a hole 902, and at least two slits (depicted as slits 904A and 904B in FIG. 9A and as slits 904C, 904D, 904E, and 904F). A diameter of the circular disc may be 14 centimetres. A radius of the hole 902 is 2 centimetres. The slits 904A-F are cut from a perimeter edge of the circular disc, and is at a predefined distance away from the hole 902. In FIG. 9A, the slits 904A-B are in a shape of an acute triangle, wherein angles of the acute triangle are depicted as an interior angle A, and exterior angles B and C. For example, the interior angle A may be 41 degrees, the exterior angle B may be 91 degrees and the exterior angle C may be 96 degrees. In FIG. 9B, the slits 904C-F are in a shape of a rectangle, wherein the slit 904C and the slit 904E are opposite to each other, and the slit 904D and the slit 904F are diametrically opposite to each other. A length and width of the slits 904C-F may be 5 centimetres and 0.7 centimetres, respectively.

FIGS. 9A-B are merely examples, which should not unduly limit the scope of the claims herein. A person skilled in the art will recognize many variations, alternatives, and modifications of embodiments of the present disclosure.

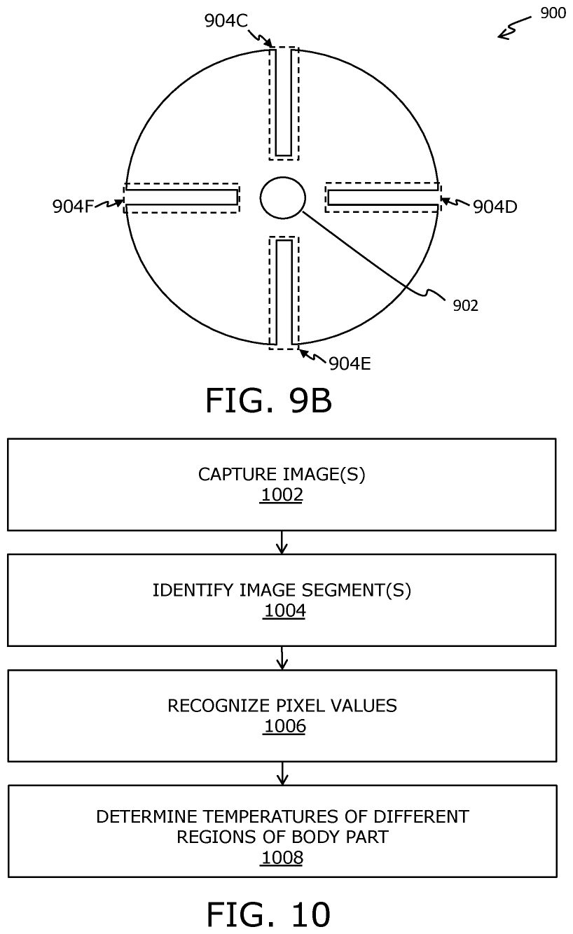

FIG. 10 is an illustration of a flowchart depicting steps of a method for determining the differential temperature in a body part, in accordance with an embodiment of the present disclosure. At step 1002, at least one image of a first side of at least one sensor device is captured. The at least one image is captured within a given time period from a time of removal of contact between the first side and the body part. At step 1004, in the at least one image, an image segment representing at least one temperature-sensitive element of the at least one sensor device is identified. At step 1006, pixel values of pixels in the at least one image segment are recognized. At step 1008, temperatures of different regions of the body part are determined, based on the pixel values of pixels in the at least one image segment, the temperatures of different regions of the body part form a differential temperature profile of the body part. It will be appreciated that prior to step 1002, the method requires providing at least one sensor device comprising a temperature sensitive material. At step 1004, an image (or each image) is identified, preferably by capturing the image using a digital camera, for example a camera of a mobile device. If the image is capture using the camera of a mobile device, and using the function inbuilt in a software application of the present invention, then step 1008 is carried out automatically by the software application, which in turn will determine and/or display the temperatures of different regions of the body part form a differential temperature profile of the body part. Alternatively, the image may be stored a mobile device comprising the software application of the present invention, and subsequently read and interpreted by the software application according to step 1008 upon a command from the user of the mobile device comprising the said software application. Furthermore, the method may comprise providing an indication of one or more abnormalities on a body part as a subsequent step. In such a case, the user will scan a unique identifier provided on the base of the sensor device. This step of scanning (providing the information to the software application) the unique identifier is essential for ensuring accuracy of measurements after reuse of the device as explained above. In particular, the device comprising thermochromic ink, may be reused for a number of times after the device is cooled to a temperature that is lower than the lower limit of the operating range of the device, which may be, for example, 25-40 degrees Celsius, or 27 to 38 degrees Celsius. The number of reuses allowed may vary per sensor device since it may slightly vary depending on handling of the device. Therefore, such number is defined by the software application based on a calibration of the expected normal temperature range of a body part. If such a normal expected temperature, for example a temperature of a body part that has not regions with different temperatures, is sensed (detected by the colours) by the material showing a colour (i.e. a pixel value) outside of a predefined calibrated range of values, the software application will provide a message that reuse should no longer be made.

The aforementioned steps are only Illustrative, and other alternatives can also be provided where one or more steps are added, one or more steps are removed, or one or more steps are provided in a different sequence without departing from the scope of the claims herein.

1. A system for determining differential temperature in a body part, the system comprising:
at least one sensor device comprising
a base having a first side and a second side opposite to the first side; and
at least one temperature-sensitive element provided on the first side of the base, wherein the temperature-sensitive element is arranged to sense temperatures of different regions of a body part of a user upon contact with the body part of the user for a predefined time period by changing it colour proportionally to the temperature of each region of the body part.
at least one camera configured to capture at least one image of the first side, wherein the at least one image is captured within a given time period from a time of removal of contact between the first side and the body part; and
at least one processor communicably coupled to the at least one camera, wherein the at least one processor is configured to:
identify, in the at least one image, at least one image segment representing the at least one temperature-sensitive element;
recognize pixel values of pixels in the at least one image segment; and
determine the temperatures of different regions of the body part, based on the pixel values of pixels in the at least one image segment, wherein the temperatures of different regions of the body part form a differential temperature profile of the body part.

2. A system according to claim 1, wherein the at least one processor is further configured to:
for each region amongst the different regions of the body part, determine whether a temperature of said region exceeds a predefined temperature threshold value; and
generate a thermal map of the body part based on the differential temperature profile of the body part, wherein the thermal map indicates any region whose temperature exceeds the predefined temperature threshold value.

3. A system according to claim 1, further comprising an infrared temperature measurement device configured to measure test temperatures of different regions of the body part, and wherein the at least one processor is further configured to receive the test temperatures of different regions of the body part from the infrared temperature measurement device.

4. A system according to claim 1, further comprising at least one user device communicably coupled to the at least one processor, wherein the at least one processor is further configured to send, to the at least one user device, at least one of: the temperatures of different regions of the body part, a thermal map of the body part, test temperatures of different regions of the body part, for presentation on the at least one user device.

5. A system according to claim 1, wherein the at least one sensor device comprises a unique identifier provided on the base, the at least one camera being configured to capture at least one identification image representing the unique identifier, wherein the at least one processor is configured to:
identify the unique identifier in the at least one identification image;
associate the temperatures of different regions of the body part that are determined to the unique identifier; and
perform one of: block future interpretation of sensed temperatures from the at least one sensor device, permit future interpretation of sensed temperatures from the at least one sensor device upon successful reuse actions being implemented.

6. A system according to claim 5, wherein the at least one processor is further configured to:
process the at least one identification image to determine at least one usage parameter, wherein the at least one usage parameter comprises at least one of: the body part for which the at least one sensor device is used, a number of times the at least one sensor device has been used, a remaining number of times of future use of the at least one sensor device, a total number of times the at least one sensor device can be used, a predefined temperature range for storing the at least one sensor device when not in use, a time period within which the at least one sensor device can be used; and
associate the at least one usage parameter to the unique identifier.

7. A system according to claim 1, wherein the at least one camera is configured to capture at least one body image representing at least the body part of the user, wherein the at least one processor is further configured to:
identify features of the body part that are represented in the at least one body image;
digitally superimpose at least one virtual object on the at least one body image for enabling correct placement of the at least one sensor device on the body part; and
send the at least one body image having the at least one virtual object superimposed thereon to the at least one user device for display thereat.

8. A system according to claim 1, wherein the at least one sensor device comprises a first sensor device and a second sensor device and the body part comprises a first body part and a second body part, wherein the first sensor device is used for determining first temperatures of different regions of the first body part of the user and the second sensor device is used for determining second temperatures of different regions of the second body part of the user, and wherein the at least one processor is configured to: for a given region of the body part, compare its corresponding first temperature and second temperature to determine a temperature difference therebetween.

9. A system according to claim 1, wherein the body part is one of: at least one breast, at least one portion of the skin, at least one testicle, at least one foot.

10. A system according to claim 9, wherein when the body part is the at least one breast, the base of the at least one sensor device has a hole, and the at least one temperature-sensitive element at least partially surrounds the hole.

11. A system according to claim 1, wherein the contact between the first side and the body part is made using at least one of: an adhesive, a supporting element upon which the at least one sensor device is arranged and which is to be placed in contact with the body part, a tying means, a clamping means.

12. A sensor device comprising:
a base having a first side and a second side opposite to the first side; and
at least one temperature-sensitive element provided on the first side of the base, wherein the temperature-sensitive element is arranged to sense temperatures of different regions of a body part of a user upon contact with the body part of the user for a predefined time period by changing colour of each region of the temperature-sensitive element, proportionally to the temperature of the each region of the body part.

13. A sensor device according to claim 12, wherein the at least one temperature-sensitive element is implemented as at least one of: a coating of a temperature-sensitive material on the first side, an element having wells in which a temperature-sensitive material is filled, a thermally-conductive covering in which a temperature-sensitive material is filled.

14. A sensor device according to claim 12- or 13, wherein the at least one temperature-sensitive material is at least one of: a thermosensitive fluid, a thermosensitive compound.

15. A sensor device according to any of claims 12-14, wherein when the body part is at least one breast, the base of the sensor device has a hole, and the at least one temperature-sensitive element at least partially surrounds the hole.

16. A sensor device according to any of claims 12-15, wherein the contact between the first side and the body part is made using at least one of: an adhesive, a supporting element, a tying means, a clamping means.

17. A method for determining a differential temperature in a body part, the method comprising:
capturing at least one image of a first side of at least one sensor device, wherein the at least one image is captured within a given time period from a time of removal of contact between the first side and the body part;
identifying, in the at least one image, at least one image segment representing at least one temperature-sensitive element of the at least one sensor device;
recognizing pixel values of pixels in the at least one image segment; and
determining the temperatures of different regions of the body part, based on the pixel values of pixels in the at least one image segment, wherein the temperatures of different regions of the body part form a differential temperature profile of the body part.
